(19) United States
(12) Patent Application Publication
(10) Pub. No.: US 2003/0229365 A1
(43) Pub. Date: Dec. 11, 2003

(54) Angled vascular anastomosis system

(76) Inventors: James G. Whayne, Chapel Hill, NC (US); Alexander Q. Tilson, Burlingame, CA (US); Charles S. Love, Santa Barbara, CA (US); Mitchell C. Barham, Menlo Park, CA (US); David P. Knight, Mountain View, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/187,655

(22) Filed: Jul. 1, 2002

Related U.S. Application Data
(60) Provisional application No. 60/387,824, filed on Jun. 10, 2002.

(51) Int. Cl. A61B 17/08

(52) U.S. Cl. 606/153

(57) ABSTRACT

Angled anastomosis devices and associated methodology are described herein. Connector and connector components as well as tools associated therewith are disclosed. The connectors are adapted to produce an angled end-to-side anastomosis at a graft/host vessel junction. A fitting alone, or a fitting in combination with a collar may be used as a connector. Each fitting may be deployed by deflecting its shape to provide clearance for a rear segment that rotates about adjoining hinge section(s) so to fit the connector within an aperture formed in a host vessel. Upon return to a substantially relaxed position, a trailing or heel segment anchors the fitting in place. The angled fitting may include additional side features for interfacing with the host vessel. The collar may include features complimentary to those of a fitting and provisions for securing the graft to the host vessel.

### CROSS-REFERENCE TO RELATED APPLICATIONS

This application claims the benefit of priority to U.S. Provisional Patent Application Serial No. 60/387,824 entitled “Angled Vascular Anastomosis System” filed Jun. 10, 2002, which is incorporated herein by reference in its entirety.

### FIELD OF THE INVENTION

This relates to producing end-to-side anastomoses, particularly in communication with coronary arteries, the aorta, the subclavian, iliacs, femoral arteries, popliteal arteries, radial arteries, mammary arteries, mesenteric arteries, renal arteries, carotid arteries, cerebral arteries, or other tubular structures. Accordingly, angled anastomosis connectors and associated devices are disclosed.

### BACKGROUND OF THE INVENTION

This invention provides devices and methods to position and secure bypass grafts at host vessel locations without having to stop or re-route blood flow for extended periods of time, which is a condition of conventional sutured anastomoses. In addition, this invention reproducibly creates angled anastomoses between bypass grafts and host vessels thereby optimizing flow dynamics through the anastomoses and mitigating risks associated with suturing, clipping or stapling the bypass graft to a host vessel, namely reduction of anastomotic opening or excessive bleeding from the puncture holes. These risks may be mitigated, in part, by features adapted to avoid bleeding at graft attachment sites and preventing the host vessel from collapsing around the incision point.

In performing cardiac bypass surgery, anastomosis sites are typically provided at a site along a patient's aorta, and another site along a coronary artery beyond a partial or complete occlusion. Alternatively, sequential “jumper” grafts may extend from a main bypass graft to individual coronary artery host vessels thereby requiring a single aortic anastomosis to accommodate multiple coronary anastomoses. As such, in-flow anastomoses are required along the main “feeder” graft and out-flow anastomoses are required to the host vessel coronary arteries. This eliminates the need for side-side anastomoses between a single graft and multiple coronary arteries when producing sequential anastomoses from a single aortic anastomosis. Producing an effective anastomosis along a coronary artery is particularly challenging. The outer diameter of a coronary artery where a distal anastomosis may be needed can range from between about 1 mm to about 4 mm in size. By way of comparison, the outer diameter of the aorta where a proximal anastomosis may be located ranges between about 20 mm and about 50 mm in size.

The relatively small size of the site for a distal anastomosis translates to greater difficulty in a number of ways. Basic surgical challenges are encountered in dealing with the smaller vasculature. Further, an interface issue is introduced. Often, particularly for connection with the smaller coronary arteries, a graft conduit will have a larger diameter than the host vessel. This may be due to the need for a larger diameter conduit to carry adequate blood flow or the result of using a saphenous vein which must be oriented so its valving allows blood to readily flow in the desired direction from the proximal anastomosis to the distal anastomosis, thereby orienting the larger end of the graft toward the distal site. For whatever reason, the mis-match in size in joining the graft to the coronary artery must be addressed. The angled anastomotic junction created by the connector embodiments of the invention accommodate this mis-match in ratio between the host vessel and graft inner diameters. In fact the angled design enables the connector embodiments to address any ratio between graft and host vessel inner diameters.

The present invention is adapted to handle these issues as well as others as may be apparent to those with skill in the art. The angled-type connectors described herein may be employed with precision and speed, resulting in treatment efficacy not heretofore possible.

The ability to convert coronary artery bypass grafting procedures and peripheral bypass grafting procedures to less invasive approaches involving small incisions and remote creation of anastomoses are particularly difficult with conventional suturing techniques and are amenable to the embodiments and approaches for the angled connectors and associated components.

### SUMMARY OF THE INVENTION

The invention includes various improvements in end-side anastomosis systems. Particularly, connectors for producing distal anatomoses are described. They each include a fitting comprising a heel section with a trailing segment that is deflectable about a hinge region to allow for placement and securing the device. Curvilinear side and forward-facing portions are preferred. Most preferably, these portions are configured to conform to the shape of a host vessel and direct the opening (incision) through the host vessel to assume the shape defined by the fitting. Such a fitting may alone serve as a connector between a host vessel and a graft provided that it includes features capable of compressing the host vessel and graft in place or otherwise maintaining close apposition between the graft and host vessel. Alternatively, the connector may comprise a fitting in combination with a collar adapted to secure a graft to the fitting and compress the graft and host vessel.

Various features for improving the deployability of a connector, hemostasis at the connector to host vessel interface, and blood flow through the anastomoses may be provided by the invention. Further, various tools for use in preparing for and creating an end-side anastomosis may comprise part of the invention. Finally, various instruments and accessories decreasing the access required to deploy the connector to enable minimally invasive surgical approaches may comprise part of the invention.

While connectors and deployment devices according to the present invention are preferably used in peripheral and coronary artery bypass grafting procedures, at a distal (out-flow) or proximal (in-flow) location, it is to be understood that the systems described herein may be used for purposes other than creating artery-to-artery or vein-to-artery anastomoses. The systems may also be used to produce anastomoses between bypass grafts and host vessels to treat other occlusions, vascular abnormalities such as stenoses, thromboses, aneurysms, fistulas and other indications requiring a bypass graft. The system of the present invention is also useful in bypassing stented vessels that have restenosed, and saphenous vein bypass grafts that have thrombosed or stenosed. Further, the invention may have other applications, such as producing arterial to venous shunts or fistulas for hemodialysis, bypassing lesions and scar tissue located in the fallopian tubes causing infertility, attaching the ureter to the kidneys during transplants, and treating gastrointestinal defects (e.g., occlusions, ulcers, obstructions, etc.).

The present invention variously includes the devices as well as the methodology disclosed. Furthermore, it is contemplated that sub-combinations of features, especially of the connector features disclosed, comprise aspects of the invention.

### BRIEF DESCRIPTION OF THE DRAWINGS

Each of the following figures diagrammatically illustrates aspects of the present invention. The illustrations provide examples of the invention described herein. Like elements in the various figures often are represented by identical numbering. For the sake of clarity, some such numbering may be omitted.

FIG. 1 shows a side view of an installed connector with a collar that secures a graft to the fitting and affixes the connector and graft assembly to a vessel wall.

FIG. 2 shows a side-sectional view of the installed connector in FIG. 1.

FIGS. 3a and 3b show side and isometric views of a formed fitting as may be used according to that shown in FIGS. 1 and 2.

FIGS. 4a and 4b show side and top views of a formed collar as may be used according to that shown in FIGS. 1 and 2.

FIGS. 5a and 5b-show side and top views of the collar in FIGS. 4a and b deflected using an external force during deployment.

FIGS. 6a and 6b show bottom views of two fitting embodiments thermally formed to accommodate different graft to host vessel inner diameter ratios.

FIGS. 6c and 6d show bottom views of two collar embodiments, along with the fitting embodiments in FIGS. 6a and 6b, that accommodate different graft to host vessel inner diameter ratios.

FIGS. 7a and 7b show top and side views of an alternative formed fitting embodiment that locates the toe flap of the graft against the interior surface of the host vessel.

FIGS. 8a and 8b show top and side views of a formed collar embodiment that cooperates with the fitting embodiment in FIGS. 7a and 7b to secure a graft to a host vessel.

FIGS. 9a and 9b show a single-piece connector embodiment.

FIGS. 9c shows the hinge locations of the connector in FIGS. 9a and 9b.

FIGS. 9d and 9e show top and side views of the connector in FIGS. 9a and 9b with a graft secured.

FIGS. 10a and 10b show the components of a loading tool used to secure a graft between a fitting and a collar.

FIG. 10c shows a perspective view of a loading tool base for use in securing a graft to the fitting and collar.

FIG. 10d shows a perspective view of a pushing tool for use with the loading tool base of FIG. 10c.

FIGS. 11a, 11b, and 11c show a side view, an end view, and a bottom view of an alternative inner frame (fitting) cartridge component of a loading tool embodiment.

FIGS. 12a to 12d show an outer frame (collar) cartridge component of a loading tool embodiment.

FIG. 13 shows an exploded view of the components of a loading tool embodiment that utilizes the inner frame cartridge in FIGS. 11a to 11c and the outer frame cartridge in FIGS. 12a to 12d.

FIGS. 14a and 14b show an exploded view and a detailed view of a deployment tool embodiment.

FIGS. 14c and 14d show side-sectional views of the deployment tool embodiment in FIGS. 14a and 14b.

FIGS. 15a and 15b show side views of the deflecting mechanisms of the deployment tool embodiment in FIGS. 14a to 14d in the released state and deflected state respectively.

FIGS. 16a and 16b show a perspective view and an end view of a repositioning tool.

FIGS. 17a and 17b show a perspective view and an end view of a removal/repositioning tool.

FIGS. 18a and 18b show a perspective view and an end view of a removal tool.

### DETAILED DESCRIPTION OF THE INVENTION

The variations of the invention discussed herein are applicable to robotic surgery, endoscopic, and other less invasive (i.e., minimally invasive) surgery. As noted above, the present invention includes variations of anastomosis connectors having features adapted to perform angled anastomoses. Anastomotic connectors, tools and associated methodology for producing in-flow (proximal) and out-flow (distal) anastomoses are described variously in U.S. and foreign patent and applications entitled, “Percutaneous Bypass Graft and Securing System”, U.S. Pat. No. 5,989,276; “Percutaneous Bypass Graft and Securing System”, U.S. Pat. No. 6,293,955; “Percutaneous Bypass Graft Securing System”, PCT Publication No. WO 98/19625; “Sutureless Anastomosis Systems”, U.S. patent application Ser. No. 09/329,503; “Sutureless Anastomosis Systems”, PCT Publication No. WO 99/65409; “Thermal Securing Anastomosis Systems” U.S. Pat. No. 6,361,559; “Thermal Securing Anastomosis Systems”, PCT Publication No. WO 99/63910; “Aortic Aneurysm Treatment Sytems”, U.S. patent application Ser. No. 09/329,658; “Aortic Aneurysm Treatment Systems”, PCT Publication No. WO 00/15144; “Additional Sutureless Anastomosis Embodiments”, U.S. patent application Ser. No. 09/654,216; “Anastomosis Systems”, U.S. patent application Ser. No. 09/730,366; “End-Side Anastomosis Systems”, PCT Publication No. WO 01/416653; “Advanced Anastomosis Systems”, U.S. patent application Ser. No. 09/770,560; “Distal Anastomosis System”, U.S. patent application Ser. No. 09/899,346; “Distal Anastomosis System”, U.S. patent application Ser. No. 09/991,469; “Improved Distal Anastomosis System”, U.S. Provisional Application Serial No. 60/333,276; and “Sutureless Anastomosis System Deployment Concepts”, U.S. patent application Ser. No. 09/927,978 and applications and patents claiming benefit hereto, all commonly owned by Converge Medical, Inc. and each of which is incorporated herein by reference in its entirety.

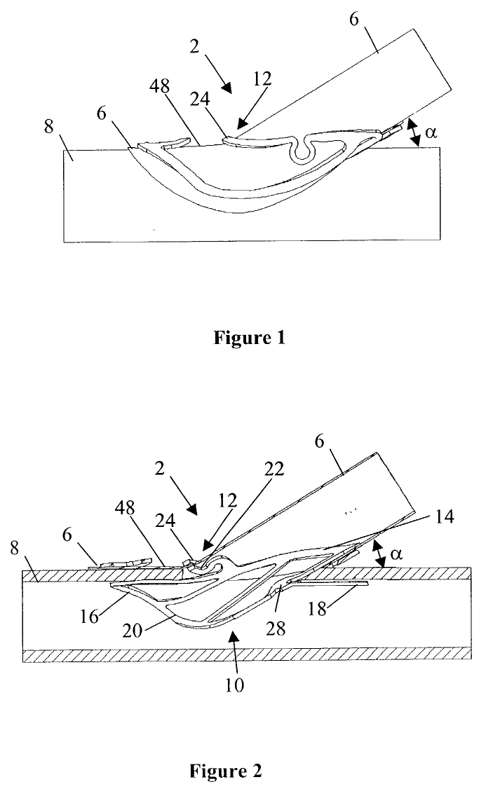

FIGS. 1 and 2 show angled anastomoses (2) formed by connectors (4) according to the present invention. Each connector (4) attaches a graft (6) to a host vessel (8). The host vessel may be any vessel or tubular structure to which a graft or other tubular structure is secured. During Coronary Artery Bypass Grafting (CABG) surgery, the host vessel is a coronary artery (Left Anterior Descending Artery, Diagonal, Circumflex, Obtuse Marginal, Right Coronary Artery, PDA, etc.), ascending aorta, subclavian artery or other vessel capable of bypassing an obstruction or stenosis by functioning as an in-flow or out-flow anastomotic junction. During Peripheral Grafting surgery, the host vessel is a popliteal artery, femoral artery, iliac artery, the aorta, carotid artery, radial artery, renal artery, hepatic artery, mesenteric artery, cerebral artery, saphenous vein, femoral vein, or other vessel that participates in bypassing an obstruction or stenosis by functioning as an in-flow or out-flow anastomotic junction. For CABG and peripheral vascular procedures, the graft (6) comprises an autologous vessel such as a saphenous vein, radial artery, left internal mammary artery, right internal mammary artery, other tissue (e.g. pericardium, submucosal, etc.) formed into a tubular structure, a synthetic graft (such as expanded PTFE or urethane derivatives), a genetically produced vessel, a donor vessel, or other tubular structure. In addition, one anastomoses' graft may function as another anastomoses' host vessel where connector are also used as in-flow anastomotic junctions to produce a series of jumper connections from a main graft to several spaced apart target conduits.

Connector Embodiments & Associated Components

The connector in FIG. 1 includes a fitting (hidden) secured to the graft and the host vessel with a collar (12). FIG. 2 shows a side-sectional view of the connector in FIG. 1. The connector in FIGS. 1 and 2 may be utilized as an outflow anastomotic junction where blood passes through the graft, past the connector, and into the host vessel where it is capable of flowing antegrade and retrograde. Alternatively, the connector in FIGS. 1 and 2 may be utilized as an in-flow anastomotic junction where blood passed through the host vessel, past the connector, and into the graft.

Referring to FIG. 2, various features of fitting (10) may be observed. First, it is noted that fitting and attached graft (6) are preferably configured so its base or body (14) is at an angle α with respect to host vessel (8). Connectors (2) are shown at approximately a 30° angle. Preferred angles for distal anastomosis range from about 20° to about 70°. A more preferable range is from about 25° to about 45°. Most preferably, they are approximately 28-30°. Because of the design of the connector, the angle helps maintain hemostasis and optimize blood flow once the anastomosis is created and retracted organs and tissue bear upon the site. Pressure created by such action will not dislodge connector (4) or kink or collapse graft (6) since the connector allows the graft (6) to extend at an acute angle such that the graft closely apposes the host vessel, and lies substantially in line with the host vessel and adjacent anatomy. In addition to improving blood-carry capability of the conduit in assuring stability of the connector, including some angle in the connector enables the manner of deployment and attachment taught below.

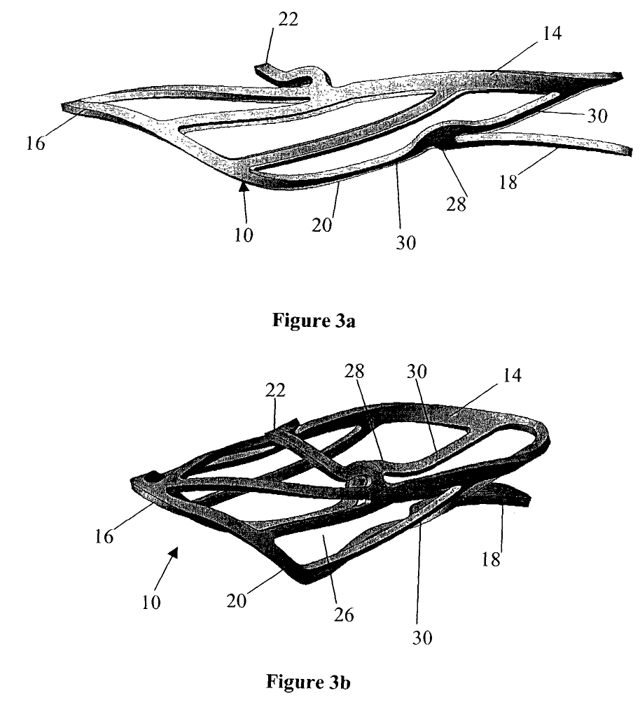

As shown in FIGS. 2, 3a, and 3b, fitting (10) includes at least a front or leading segment (16) and a rear or trailing segment (18). When situated to form an anastomosis, these segments lie approximately in line with host vessel (8). So-placed, they prevent removal of the connector from the host vessel. Optional lateral or side portions (20) may also aid in this regard. This is especially the case when forming an anastomosis with a very small diameter vessel (such as a 1 to 4 mm inner diameter host vessel). Furthermore, lateral portions (20) extend beyond the plane of the trailing segment (18) and interconnect with the leading segment (16) to ensure the host vessel tissue about the opening through the host vessel is completely captured around the anastomosis thereby ensuring a physical barrier to leakage. This may be true irrespective of the size of host vessel (8). The one or more lateral portions (20) on each side of fitting (10) also provide a smooth transition between the leading and trailing portions of fitting (10) to facilitate insertion of the connector through an opening in the host vessel and help moderate or alleviate trauma to the interior of the host vessel (8) while deploying the connector.

A lateral portion may be provided integrally with a form providing at least part of leading segment (16) and trailing segment (18). As described above, this continuous coverage ensures complete tissue capture between the fitting (10) inside the host vessel and the collar (not shown) outside the host vessel. Complete coverage ensures hemostasis at the vessel to graft interface.

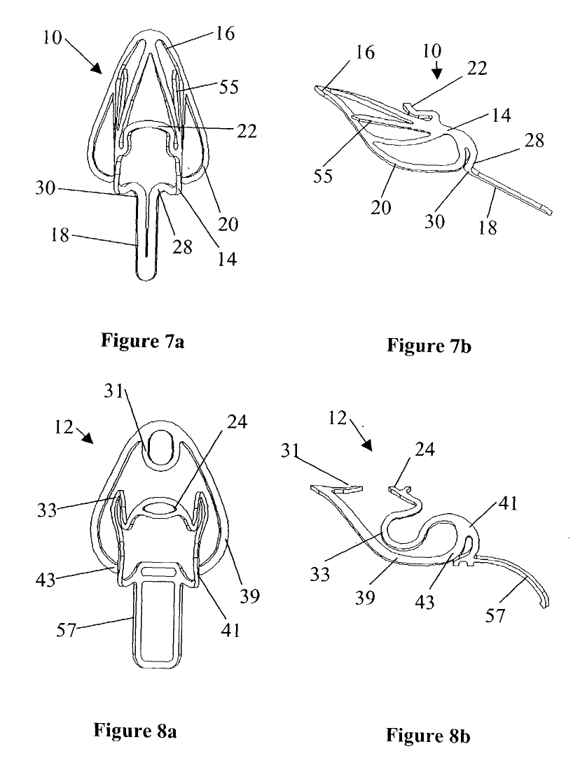

As shown in FIGS. 3a and 3b, additional optional features of fitting (10) include tabs or latches (22) to assist in securing graft (6) and/or optional collar (12). Such tabs may be oriented to grip graft (6) as shown in FIG. 2. One or more tabs may also be adapted to form a locking interface with one or more complementary tabs or latches (24) optionally included in collar (12). Also, the height or amount of material incorporated in the base of the fitting may be varied. In order to utilize as little material as possible to join the various segments, base (14) may be provided by a narrow band of material as shown in FIGS. 3a, 7b or otherwise. To achieve proper relative placement of these features, base (14) may be curved or undulate.

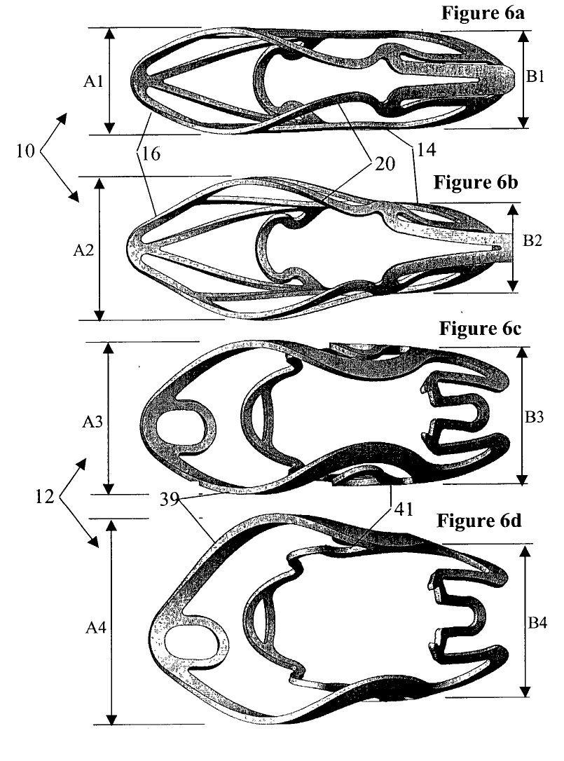

As shown in FIG. 3b, the connector opening (26) may have an ovalized or elliptical opening to the anastomosis, or may have a circular bore. As will be discussed below, the connector is preferably fabricated from a raw tube that is laser cut into the desired pattern and thermally formed into the desired resting configuration as shown in FIGS. 3a and 3b. This inherent profile may be altered by closing the width between opposite sides of the lateral portions (20) and/or base (14) causing the connector to assume an ovalized profile with the major axis extending from the leading segment (16) towards the trailing segment (18) and the minor axis perpendicular to the major axis, as shown in FIG. 6a. Configuring fitting (10) with an ovalized opening (26) may be useful in providing an interface to a smaller host vessel. As shown in FIG. 6a, ovalizing the profile at the lateral portions (20) to a width, A1, while maintaining the profile of the base (14) to a width, B1, provides a manner in which to account for the optimal transition in the size difference between a smaller diameter host vessel and what is often a larger diameter opening of the graft by transitioning the geometry change from the ovalized anastomotic junction cross-section to the more circular graft cross-section. In this case A1≦B1. For example, a 30 degree, 3 mm connector having B1=0.117″ and A1=0.110″ is capable of transitioning a graft with an inner diameter from 3 mm to 5 mm to a host vessel with an inner diameter from 2 mm to 4 mm. A 30 degree, 3 mm connector having B1=0.117″ and A1=0.080″ is capable of transitioning a graft with an inner diameter from 3 mm to 5 mm to a host vessel with an inner diameter from 1.25 mm to 2.5 mm. The ovalization increases the available perimeter to accommodate a host vessel without having to alter the diameter of the connector. Instead, a connector is lengthened by ovalizing to accommodate smaller host vessels without having to change the diameter of the base and/or graft. Ovalizing the connector is an acceptable alteration in connector geometry since only the size of the arteriotomy made in the host vessel need be lengthened to fit the connector in place.

The angled connector geometry provides a further enhancement in that a single version accommodates a wide range of graft diameters. By angling the graft relative to the host vessel, the cut end of the graft, which defines the graft toe (48) and the angle the graft extends from the connector may be modified to produce a cross-section that matches the specific connector size.

As shown in FIG. 6b, the separation between the lateral portions (20) of the fitting (10) may be increased, A2, such that it exceeds the diameter, B2, of the base (14) to enable transitioning a larger diameter host vessel to a smaller diameter graft. This is particularly relevant when using the angled connector as an in-flow anastomotic junction between a large vessel (such as the aorta, iliac, subclavian, carotid artery, femoral artery, or other supplying vessel) and a smaller diameter graft.

As shown in FIGS. 6c and 6d, the collar profile matches that of the fitting to accommodate for the disparity in size between the host vessel and graft, if any. The collar of FIG. 6c matches the profile of the fitting embodiment in FIG. 6a such that A3<B3 to apply compression against the host vessel and graft when the host vessel diameter is equal to or smaller than the diameter of the graft. Similarly, the collar of FIG. 6d matches the profile of the fitting embodiment in FIG. 6b such that A4>B4 to accommodate larger host vessel diameters compared to the graft.

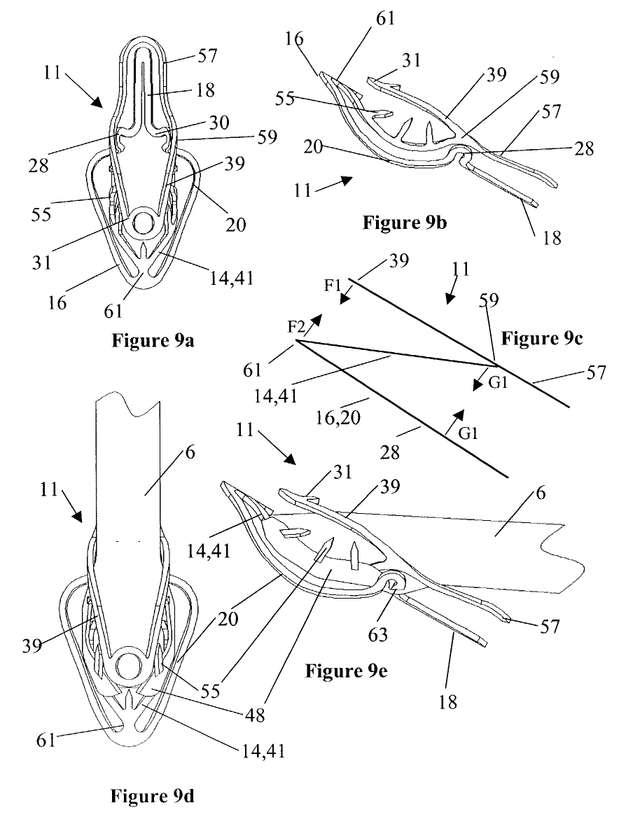

Features that are preferable for fitting (10), in addition to the basic leading and trailing segment configuration, are found in connection with a hinge section (28), shown in FIGS. 2, 3a, 3b, 7a, 7b, 9a, and 9b. Hinge section (28) may be provided in a number of configurations. However, the configurations serve the same purpose. Each of the variations shown and described allow trailing segment (18) to be displaced sufficiently to clear the host vessel wall for insertion of the connector into the host vessel by significant torsional deflection of areas between trailing segment (18) and fitting body (14). In the fitting variations shown in FIGS. 2, 3a and 3b, a pair of torsion sections (30) are presented on each side of trailing segment (18). In the fitting variations shown in FIGS. 7a, 7b, 9a, and 9b, a single torsional section (30) is presented on each side of trailing segment (18).

To displace trailing segment (18) sufficiently, the rotation about torsional sections accounts for a substantial amount of the displacement required of trailing segment (18). The additional displacement arises from bending of the trailing segment (18) relative to the junction between the trailing segment and the torsional sections.

Such dual action provides for certain advantages; namely, upon forward deflection of trailing segment (18), i.e., deflection of trailing segment (18) towards leading segment (16), the lateral portions connected to torsional sections are caused to be drawn or flexed inward. This action facilitates introduction of connector (4) into host vessel (8) by clearing portions that could otherwise interfere with entry. In addition, the design of the embodiment in FIGS. 2, 3a and 3b has a pair of torsional sections on each side of the trailing section, one integrated with the base (14) and an opposite extending one integrated with the leading section (16). The embodiment in FIGS. 2, 3a and 3b has the trailing section (18) cut from the base (14) and deflected approximately 30 degrees in its resting configuration. As such the trailing section (18) is integrated with both the base and the leading section to provide a continuous band of support throughout the anastomosis along the interior surface of the host vessel, increase the resistance to deflection once the connector is deployed, and provide a wedge between the trailing section (18) and the base (14) capable of increasing the compression forces that the trailing section (18) and the base (14) exert against the graft and the host vessel to ensure hemostasis at the heel of the anastomosis.

The embodiment in FIGS. 7a, 7b, 9a, and 9b similarly has the trailing section (18) cut from the direction of the base (14) however, the base in this embodiment has been shortened and extends from just adjacent to the trailing segment (18) to the leading segment (16). Therefore, the trailing section (18) still provides a continuous band of support throughout the anastomosis but the base does not inhibit the ability to extend the graft at a more acute angle than 28 to 30 degrees.

Turning now to the features of collar (12), FIGS. 1, 4a, 4b, 5a, and 5b illustrate desirable features of this part of connector (4). One purpose of collar (12) is to secure the graft (6) and host vessel to fitting (4) and ensure the graft produces a gasket against the host vessel throughout the periphery of the anastomosis to ensure hemostasis. As noted above, optional collar tab(s) or latch(es) (24) may assist in this regard by interfacing with optional fitting tab(s) or latch(es) (22). Also, collar (12) may be resiliently biased against graft (6) and host vessel to hold it to fitting (10). Further, expansion spring members (35) may be provided to enable expanding the diameter of the collar for placement around the fitting and returning the collar towards its preformed configuration once positioned to ensure a secure fit of collar (12) about fitting (6).

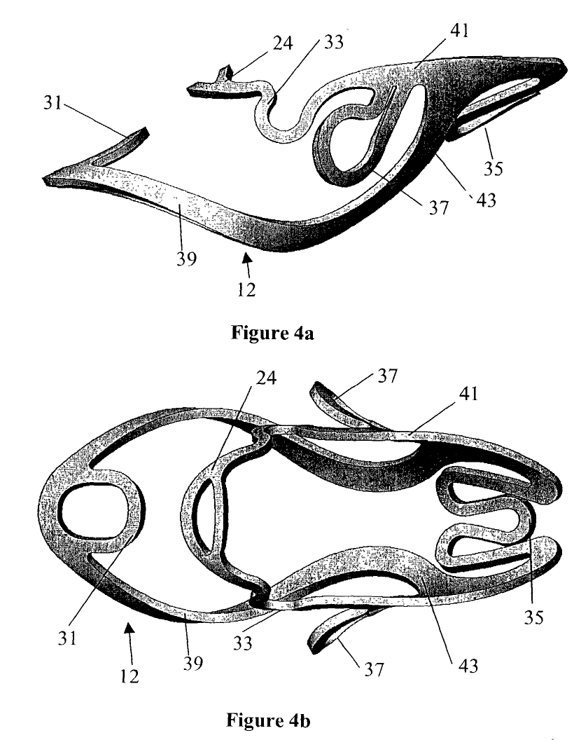

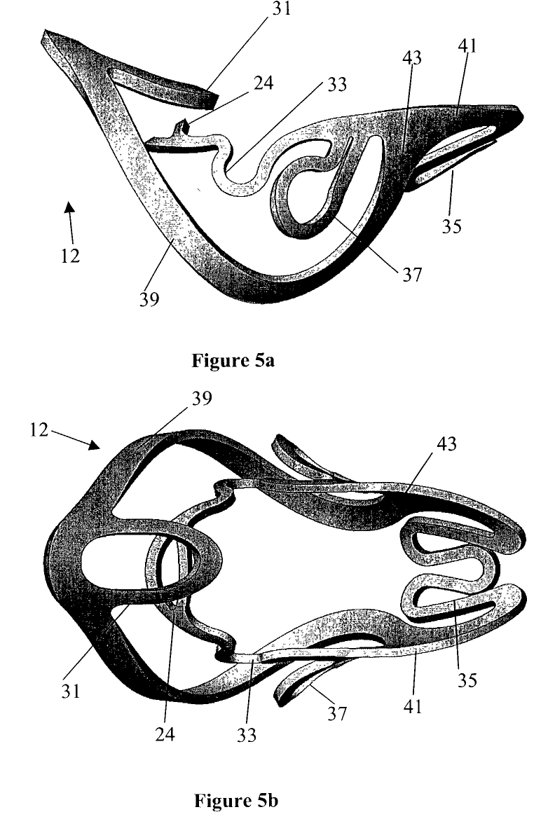

The expansion spring members (35) in the embodiment in FIGS. 1, 4a, 4b, 5a, and 5b incorporate a vertical undulating pattern, which enlarges as the collar is expanded from its resting diameter towards an enlarged geometry. This expansion spring configuration has a middle undulation and two side undulations. The length of the middle undulation is shorter than that of the side undulations (approximately ½ to ¼ shorter), and the widths and wall thicknesses are the same so enlarging the expansion spring first separates the side undulations without altering the middle undulation and only after substantial enlargement of the side undulations does the middle undulation separate. This helps orient the trailing segment (18) of the fitting (4) relative to the expansion spring (35) while loading the fitting and graft to the collar. Another alignment feature shown in FIGS. 4a, 4b, 5a, and 5b are short protrusion extending from the junction between the side undulations and the middle undulation that orients the trailing segment (18) relative to the expansion spring (35) and maintains that orientation during manipulation of the connector.

This expansion spring embodiment also enables lengthening the distance from the tab or latch (24) of the collar and the location on the expansion spring to which the trailing segment of the fitting abuts. This facilitates securing the collar to the fitting around the graft by locating the tab (24) of the collar beyond the tab (22) of the fitting without having to engage and dramatically pull tab (24) past the tab (22). Upon releasing the external force deflecting the collar, the expanding spring members recoil towards the undulating pattern urging the collar towards its resting, smaller diameter configuration thereby engaging the tab (24) of the collar to the tab (22) of the fitting and compressing the collar against the base (14) of the fitting.

Preferably, the distal band (39) of the collar (12) extends completely around the anastomosis from the heel to the toe to overlap or interface with corresponding lateral features (20) of a complimentary fitting (10) to form a complete seal at an anastomosis site. Likewise, the shape of the bore of the collar as shown in FIGS. 6c and 6d should complement that of the fitting (e.g. FIGS. 6a and 6b respectively). In instances where the fitting has a circular bore (26), at least a mating portion of collar (12) should be substantially circular as well. In instances where fitting bore (26) is ovalized, a corresponding shape should be utilized in collar (12). For instances where the fitting is tapered in geometry from a circular profile at the graft to an ovalized or enlarged profile at the anastomotic junction, the collar should also possess such features. The distal band (39) is secured to the base of the collar at the heel to enable deflecting the distal band (39) upward during deployment, as shown in FIG. 5a. The semicircular nature of the distal band (39) causes the distal band to buckle outward as it is deflected with a deployment tool, as shown in FIG. 5b. This provides separation between the distal band (39) and the lateral sections (20) of the fitting to ensure host vessel tissue can enter this gap such that once positioned, the distal band may be released thereby compressing the graft and the host vessel against the fitting's leading section and lateral sections ensuring complete hemostasis around the periphery of the anastomosis.

Another feature of the collar (12) embodiment shown in FIGS. 1, 4a, 4b, 5a, and 5b involves side spring loops (33). These side spring loops (33) enable axial extension of the tab (24) during loading of the collar over the graft and the fitting to enable placing the tab (24) of the collar into engagement with the tab (22) of the fitting without requiring significant manipulation of the fitting and collar. The utility of the side spring loops (33).is diminished if the expansion spring enables adequate lengthening of the tab (24) relative to the expansion spring yet provides additional axial lengthening of this dimension during loading of the graft and fitting to the collar.

Ears, shown in FIGS. 4a, 4b, 5a, and 5b provide an engagement point for pins of a deployment tool to stabilize the connector during deployment or a loading tool to manipulate the collar during placement of the graft and/or locking of the fitting to the collar. The ears may or may not be thermally formed in a radially outward configuration such that the deployment tool and/or loading tool pins may be readily inserted from the top, front, or rear, depending on the location of the pins on the deployment tool.

The collar embodiments in FIGS. 4a, 4b, 5a, and 5b also incorporate a grasping loop or link (31) that provides an exposed edge which the deployment tool may engage and deflect the distal band (39) relative to the base of the collar. This facilitates engagement and removal of the deployment tool relative to the collar.

Whether prepared in connection with a collar or not, connector (4) is preferably installed at an anastomosis site as shown in FIG. 2. Here, it may be observed that graft toe (48) preferably overlaps host vessel (8). A heel portion (62) may abut, overlap host vessel (8) or leave a slight gap.

When connecting a graft to a small diameter host vessel, the graft toe (48) preferably resides along the exterior surface of the host vessel so it doesn't substantially reduce the cross-sectional area of the host vessel. When a connector is provided with a collar (12), the visible result will resemble that in FIG. 1. Still, one preferred relation of graft (6) to host vessel (8) remains similar to that shown in FIG. 2, depending on the fitting configuration selected. Alternatively, the graft toe (48) may be oriented such that it resides along the interior surface of the host vessel and the host vessel overlaps the graft toe. This is an especially suitable alternative when the connector is attaching a graft to a larger diameter host vessel.

The function of the connector (as an in-flow anastomotic junction or out-flow anastomotic junction) also impacts the location of the graft toe (48) (e.g. inside the host vessel, and/or outside the host vessel). Other aspects of the anastomotic junction also impact the location of the graft toe. For example, when securing a graft to a host vessel having a large wall thickness (e.g. aorta), the graft toe (48) is preferably located along the interior surface of the host vessel so the thick cut end of the aorta is not exposed to blood flow. As such, flow disruptions are avoided by ensuring a smooth transition from the graft to the host vessel. When everting the tissue to minimize the metal exposed to blood, the graft toe is preferably located along the interior surface of the host vessel therefore the cut end of the graft and host vessel are isolated from blood flow. As shown in FIGS. 9d and 9e, the cut/beveled end of the graft toe readily everts around the toe of the fitting (10); the cut bevel easily wraps around the slightly curved cross-section of the leading segment (16) by taking opposite free edges of the cut tissue and pulling them around opposite sides of the leading segment and securing them in place by use of pins (55) and/or compressing them between two components as shown in FIGS. 9c and 9d. On the contrary, the side of a host vessel is extremely difficult to evert because all edges of the tissue are constrained so the only way to evert is to over-stretch the tissue which results in unwanted damage.

FIGS. 7a and 7b show an alternative fitting embodiment (10) that along with collar embodiment shown in FIGS. 8a and 8b produce a connector capable of producing an in-flow anastomotic junction and/or an anastomosis having a host vessel to graft inner diameter ratio >>1. As shown in FIG. 7a, the separation between lateral portions (20) is increased to accommodate the larger host vessel while the separation between the sides of the base (14) accommodate the smaller graft. As described above, a latch or tab (22) on the fitting mates with the corresponding latch or tab (24) on the collar (see FIGS. 7a and 8a). The trailing segment (18) in this embodiment is designed to penetrate through a small puncture in the heel portion of the graft just proximal to the end of the incision (described below). This secures the heel of the graft to this fitting embodiment because the stem region at the heel of the fitting is non-existent. Pins (55) may be used to hold the toe region of the graft against the fitting during insertion through the arteriotomy ensuring the graft toe region resides against the interior surface of the host vessel. The collar incorporates a heel segment (57) to account for the elimination of the wedge with this embodiment. A slot in the heel region accommodates insertion of the trailing segment (18) to lock the collar to the fitting at the heel. As previously stated, tab (24) may be locked to tab (22). Side springs (33) enable extension of tab (24) beyond tab (22) during loading and return towards its resting configuration when the external, extension force is removed thereby locking tab (24) to tab (22). A distal band (39) matches the leading segment (16) and lateral portions (20) of the fitting to provide compression around the anastomosis. A grasping loop (31) enables deflecting the distal band (39) as will be described below. It should be noted that this embodiment may be modified to accommodate host vessel to graft inner diameters ≦1 by thermally forming the lateral portions (20) of fitting and separation of distal band (39) of collar to accommodate host vessel inner diameters smaller than or equal to the graft inner diameter.

FIGS. 9a and 9b provide an all-in-one connector embodiment that incorporates the fitting and collar functions into a unitary connector. This unitary connector (11) incorporates a leading segment (16) that defines lateral portions (20) which are integrated to a trailing segment (18). As described in FIGS. 7a and 7b above and shown in FIG. 9e, the trailing segment (18) is placed through a puncture (63) in the heel of the graft just beyond the incision through the graft that produces the graft toe. This locks the graft to the connector at the heel region. Leading segment (16) produces a hinge (61) to base (14,41) that enables deflecting the leading segment, lateral portions, and trailing segment while placing the graft toe between the lateral portions (20) and base (14,41). Once positioned, the external force deflecting the lateral portions is removed allowing the lateral portions to return towards their preformed shape compressing the graft toe (48) between the lateral portions (20) and the base (14,41). A second hinge (59) integrates the distal band (39) and the heel segment (57) to the base (14,41). The distal band (39) is deflected during deployment, as described below, to provide a separation that host vessel tissue may enter for compressing the graft and host vessel between components of the connector. The heel segment (57) compresses the host vessel against the trailing segment (18) to maintain position of the connector in the host vessel and stabilizes the graft at the heel of the anastomosis. Pins (55) may be used to evert the graft toe (48) to lock the graft in place. The pins (55) may be used when the compression force between the lateral portions (20) and the base (14,41) about hinge (61) is not adequate to lock the graft to the connector or when the operator wants to isolate the cut end of the graft from blood flow. FIGS. 9d and 9e show the unitary connector (11) with a graft toe (48) clamped between the lateral portions (20) and the base (14,41) and everted over pins (55). FIG. 9c shows the compression forces used to lock the graft and host vessel to the unitary connector. Forces (F1, F2, G1, and G2) may be optimized by altering the stiffness and/or spring constants of hinges (61 and 59) to ensure the graft and host vessel are captured by and locked to the unitary connector (11).

Angled Anastomoses Procedure and Accessory Devices

Now that many of the device features of the invention have been described, the process associated therewith is set forth in the order in which it is preferred that a surgeon or surgical team take action to perform a coronary bypass procedure, peripheral bypass procedure, or other procedure associated with creating anastomoses between tubular body structures during surgical, minimally invasive, endoscopic, robotic, catheter-based, or a combination of these approaches. Variation of this procedure is, of course, contemplated. Furthermore, it is to be understood that the devices described herein may be used outside of this context.

This being said, after opening a patient and taking a measurement between intended target sites for in-flow (proximal) and out-flow (distal) anastomoses, a graft member (6) of sufficient length is obtained. Typically this will be a saphenous vein. Alternately, another harvested vessel (such as the left internal mammary artery, right internal mammary artery, radial artery, or other autologous vessel), a synthetic graft (e.g. ePTFE, urethane, etc.), non-vascular autologous tissue (e.g. pericardium, submucosa, etc.), a genetically engineered tubular structure, or a donor tissue may be used as a graft.

Especially in the case where an organic member is used, the vessel will be sized to determine the appropriate connector size. This is preferably done with reference to the inner diameter of the graft by inserting pins of increasing size (e.g. by 0.25 mm increments) until the graft no longer easily fits over a given pin. The size of the largest pin over which graft easily fits over sets the inner diameter of the graft. Alternatively, a “go/no-go” gauge may be used where a single connector covers a wide range of graft inner diameters. The “go/no-go” gauge would have a minimum inner diameter and a maximum inner diameter at which the inner diameter of the graft should reside to be used with the specific connector configuration.

Next, a connector for producing an anastomosis at a desired angle, and having an appropriate size is chosen. The size of fitting (10) and optional collar (12) covers a range of graft inner diameters and is preferably chosen by matching the first incremental size over the inner diameter of the graft to a chart of connector sizes that accommodate the measured graft diameter. It is contemplated that connector component sizes may be sized to fit grafts of a diameter from about 2 mm to about 6 mm progressively, at 0.5 mm to 2.0 mm increments. The acute angle of the connector embodiments enables a specific connector size to accommodate a wide range of graft sizes because the graft is oriented at an angle relative to the connector bore and this relationship may alter based on the size matching between the graft and the connector. For example, a 3 mm diameter connector has been demonstrated to accommodate graft inner diameters between 3 mm and 5 mm without constricting the lumen of the graft or otherwise adversely affecting the transition from the graft to the host vessel with respect to flow barriers or disruptions.

Once appropriately sized connector components are chosen, a graft is skeletonized 10 mm away from the end to be used in connection with the anastomosis. This may be accomplished by holding the adventitia tissue away from the graft with forceps and removing selected portions with Potts or Dissecting scissors.

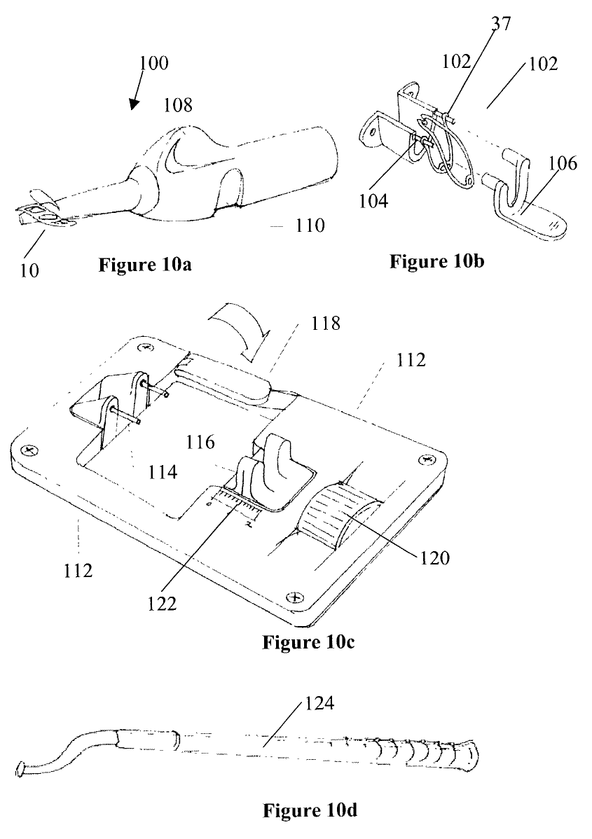

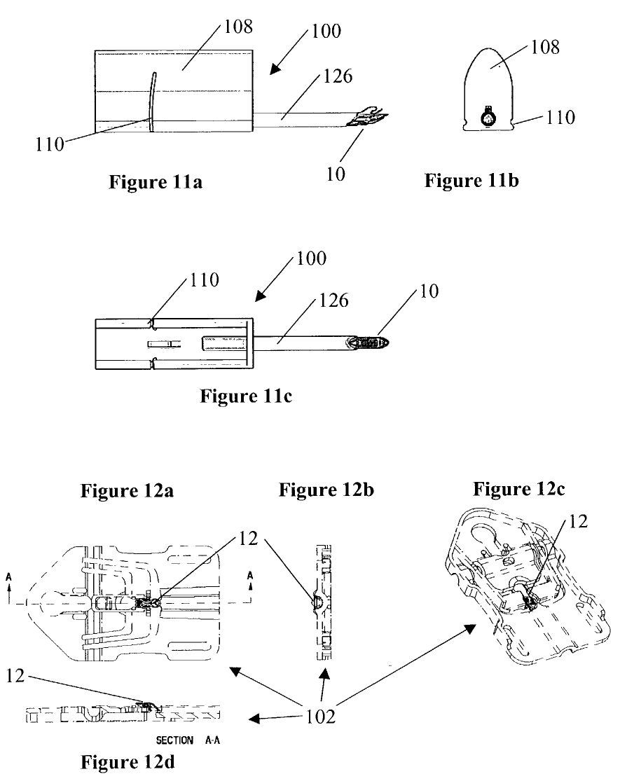

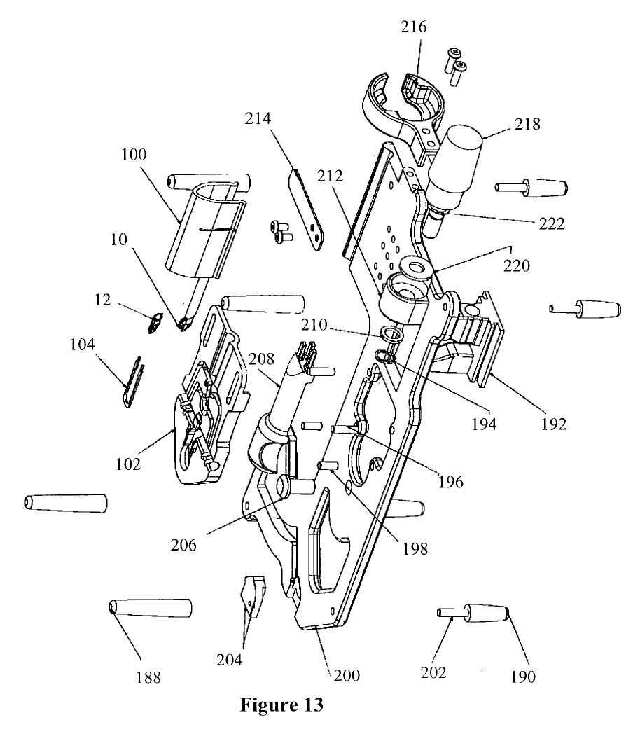

At this stage, graft (6) is passed through the collar (12), which has already been expanded to facilitate advancing the graft. The collar (12) may be housed on a loading cartridge (see FIGS. 10b, 12a to 12d) which, when attached to the loading tool base (see FIGS. 10c and 13), may be expanded by spreading the ears of the collar (12) apart thereby expanding the collar (12) at the expansion spring and providing an enlarged lumen through which to pass the graft. The loading cartridge (102) may contain a flex region, an interlock, and pins (104). The pins (104) are used to stabilize the collar (12) during shipment and expansion on the loading tool. A mating insert (106) may be used to stabilize the collar (12) relative to the outer frame cartridge (102) during shipping; this insert (106) is removed and disposed prior to placing the outer frame cartridge. The interlock enables temporarily securing the loading cartridge to the loading tool (112) during placement of the graft and latching of the fitting. The flex region provides an integrated hinge through which the loading cartridge thus the collar may be expanded. A lever (118) may be used to manually expand the collar, as shown in FIG. 10c; alternatively, as shown in FIG. 13, the collar automatically expands as the outer frame cartridge is locked to the loading base.

Advancing graft (6) through collar (12) may be accomplished with an elongate, low profile clamp or forceps to pull graft through the expanded collar. Once the graft is positioned, an incision from the free end of the graft is created to define the graft toe (48). The length of this incision depends on the diameter of the connector and the angle of the anastomosis. For a 30 degree, 3 mm connector, a 9 to 10 mm incision is created to define the graft toe (48). The graft toe (48) must completely cover the leading segment (16) of the fitting (10) and extend around the lateral portions (20). This graft toe (48) provides the interface at which the cut edges of the host vessel are clamped thereby ensuring hemostasis.

Then, the fitting (10) is inserted through the cut end of the graft until the trailing segment (18) of the fitting abuts the expansion spring (35) of the collar. This ensures that the graft is completely captured between the fitting and the collar, which is essential to ensuring hemostasis at the anastomosis. Once in place about fitting (18), graft (6) may be trimmed to more closely conform to the shape of connector elements, particularly the distal band (39) of the collar (12).

In placing fitting (10) into graft (6), it is to be set in relation to collar (12) in a complementary manner. When optional tabs (22) and (24) are provided, these features can easily be used to help align a fitting and a collar relative to each other. Either way, once collar (12) and fitting (10) are properly aligned, tabs and/or locking features (36) are engaged with each other, collar (12) is released onto graft (6), and a final check is made to ensure accurate component placement and graft coverage.

The loading tool primarily facilitates these steps by utilizing the design of the collar and fitting to minimize the amount of manipulation required to engage the tabs and lock the collar to the fitting about the graft. Once the outer frame cartridge (102) is placed onto the loading tool (112), e.g., at pins (114), it is expanded so the graft may be inserted through the bore of the collar. After cutting the incision in the graft the inner frame cartridge (100) is used to advance the fitting into the cut end of the graft such that the trailing segment (18) of the fitting is oriented into engagement with the expansion spring (35) of the collar. As shown in FIGS. 10a, and 11a to c, different variations of the inner frame cartridge (100) incorporates a snap (110) and a handle (108) to direct the insertion path of the fitting (10), which is placed on the end of a positioning shaft (126), such that the base (14) of the fitting passes into the cut end of the graft and under the expansion spring (35) of the collar while the trailing segment (18) of the fitting resides outside the graft and expansion spring. Once the trailing segment (18) is appropriately positioned, the inner frame cartridge is snapped into engagement with the loading tool at dock (116). Then the inner frame cartridge is advanced using a shaft dial (120 or 218) thereby advancing the fitting relative to the collar. An indicator gauge (122) may be placed upon the loading tool (112) to indicate the distance advanced by the fitting. The expansion spring stretches at the side undulations causing the distance between the tabs of the collar and fitting to shorten. Once the inner frame cartridge is fully advanced, the tab of the collar extends beyond the tab of the fitting. It has been demonstrated that 0.070″ to 0.150″ extension of the collar at the expansion spring using the fitting places the tab (24) of the collar beyond the tab (22) of the fitting. The loading tool is rotated 180 degrees and a pusher (124) (see FIG. 10d) is used to apply downward pressure against the tab or latch of the fitting while the shaft dials of the loading tool are used to retract the inner frame cartridge allowing the expansion spring to return towards its resting undulating shape and engaging the tabs about the graft. At this point the connector and graft assembly is complete and ready for deployment.

The loading tool embodiment shown in FIG. 13 also includes features to stabilize the deployment tool while placing the connector assembly into the deployment tool and deflecting the distal band (39) of the collar (12) and the trailing segment (18) of the fitting (10).

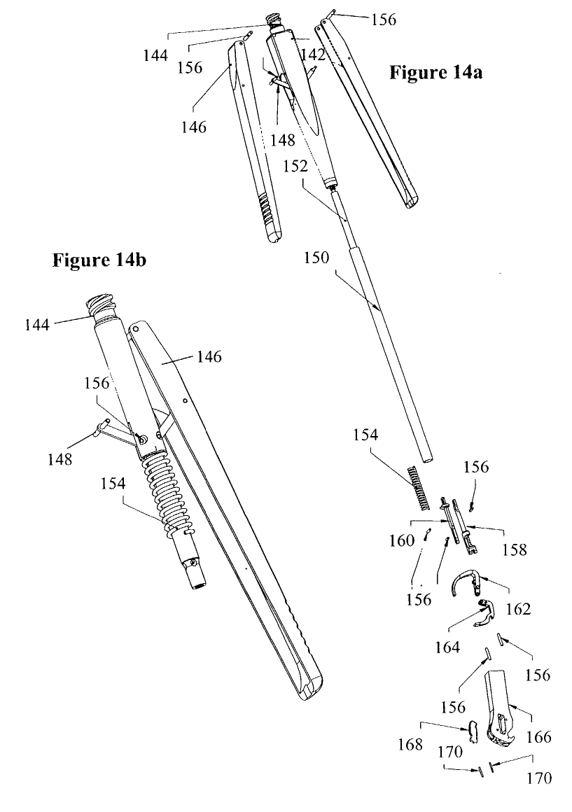

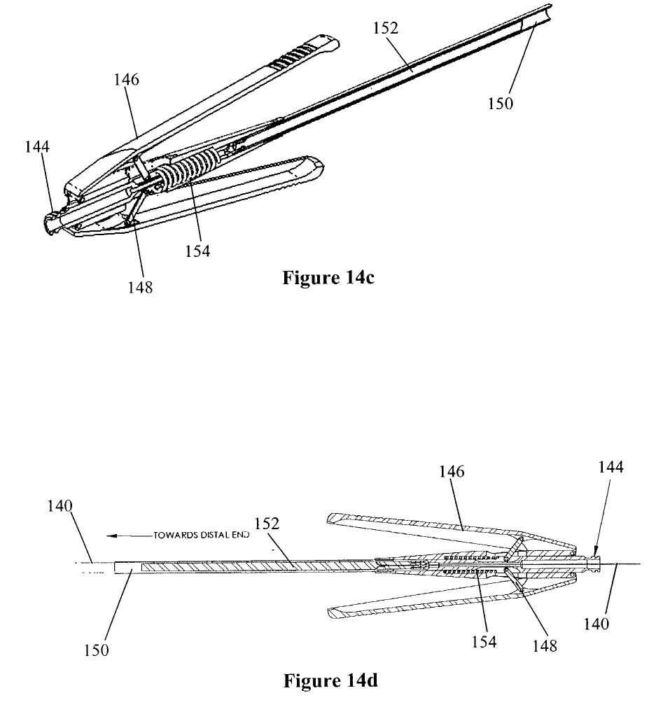

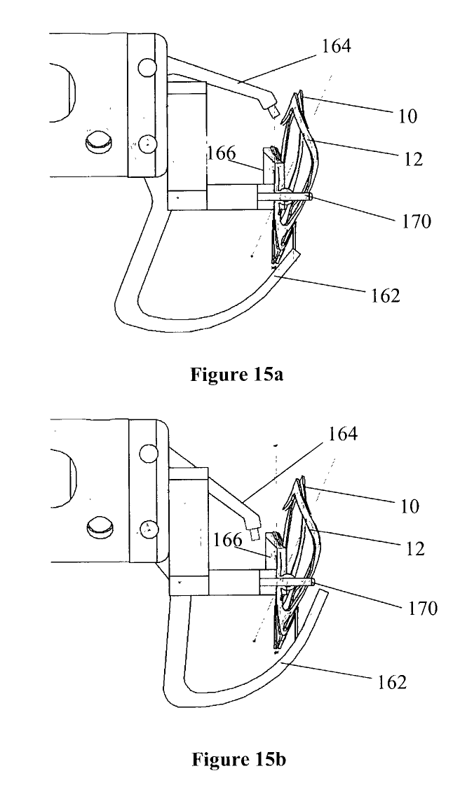

It is preferred that connector (4) be set and prepared for deployment within a deployment device, as shown in FIGS. 14a and 14b, before taking invasive action at the target site for an angled anastomosis. Regardless, an angled anastomosis site is prepared by creating an initial puncture, for instance, with the tip of a number 11 blade scalpel. Next, this opening is preferably extended longitudinally with scissors to about 3 mm to 7 mm in length depending on the connector size and anastomosis angle. Most often, a longitudinal slit of about 5 mm is preferred for a 30 degree, 3 mm connector. Scissors are advantageously provided in connection with an instrument. Otherwise, standard Potts scissors may be used. In one arteriotomy (or venotomy) instrument embodiment, a marker pen is used to place biocompatible ink on a marking instrument with a specified length and the marking instrument is used to tattoo an identifier as to the desired incision length. This helps direct the operator to cut the incision to the appropriate length without requiring the use of a specific blade instrument designed to only create the desired incision with a single actuation.

The deployment tool in FIGS. 14a to 14d, and 15a and 15b incorporated pins (170) that engage the ears (37) of the collar. This provides stabilization of the connector relative to the deployment tool and provides a reference from which to deflect the distal band (39) of the collar. It should be noted that the deployment tool may alternatively incorporate a clamping or other grasping mechanism to engage the base of the collar and/or fitting without having to penetrate components of either the collar or fitting. One such component is a stabilization platform (166) incorporated in the deployment tool and configured to engage the front and/or lateral surface of the connector to maintain the position of the connector during deployment. A combination of stabilization platform (166) and pins (170) are used in the embodiments shown in FIGS. 14a to 14d, and 15a and 15b.

The deployment tool also incorporates a toe deflector (164) and a heel deflector (162), which engage the elliptical loop (31) to deflect and release the distal band (39) of the collar and the trailing section (18) of the fitting during deployment. FIG. 15a shows the toe deflector (164) and the heel deflector (162) in the loading or release state. FIG. 15b shows the toe deflector (164) and the heel deflector (162) in the actuated state, ready for deployment of the connector. It should be noted that in FIG. 15b, the components of the connector are not shown deflected; in operation, movement of the toe deflector and heel deflector cause their counterparts on the connector to correspondingly deflect for deployment.

Once deployed, the heel deflector (162) and toe deflector (164) are released enabling the trailing section (18) of the fitting and the distal band (39) of the collar to return towards their resting configuration causing the tissue (host vessel and graft) residing between the fitting and the collar to be compressed, like a gasket, and ensure hemostasis at the anastomosis. It should be noted that the toe deflector (164) and the heel deflector (162) may be actuated simultaneously; the toe deflector may be offset from heel deflection to enable full deployment of the trailing section of the fitting prior to full release of the distal band of the collar; or may be operated independently.

With the trailing segment and the distal band deflected into the deployment configuration, connector (4) is positioned into the host vessel. This is preferably performed by inserting the leading section (16) through the arteriotomy (or venotomy if the host vessel is a vein), and then advancing the lateral features (20) of fitting (10) as may be provided. Deflected trailing segment (18) is then advanced through the heel end of the arteriotomy and into host vessel (8); then the trailing segment (18) is released by actuating the deployment tool towards its resting configuration, as shown in FIG. 2, in order to secure the connector. Particularly in those variations of the invention as described above where movement of trailing segment articulates side portions (20), movement of trailing segment (18) to an host-vessel engaging position will also cause affected side portions (20) to engage the sides of host vessel (8) to maintain connector (4) in place.

In instances when a collar (12) is used in connector (4), it is also released to compress toe portion (48) of graft (6) against host vessel (8). Release of collar (12) may also result in compressing graft (6) against portions of host vessel (8) opposed by lateral fitting portions (20), especially when the lateral portions are integrated with the trailing segment.

The deployment tool embodiment shown in FIGS. 14a to 14d enables offsetting the movement of the toe deflector (164) relative to the heel deflector (162) with a single actuation mechanism. This offset facilitates full release of the trailing segment (18) prior to release of the distal band (39) of the collar with a single handle actuation to provide operator control of the connector release. As such the trailing segment (18) may be fully released so the operator can confirm its position within the host vessel, ensure the sides of the incision through the host vessel are appropriately positioned around the lateral portions (20) of the fitting, and/or de-air the graft prior to releasing the collar distal band (39).

The embodiment in FIGS. 14a to 14d includes two handle segments (146) rotatably connected to a handle block (142) at a proximal end directly with pins (156). The handle segment (146) is secured to linkages (148) that pass through slots in the handle block (142) at a mid-section and are secured to a rod (152) that contains a luer end (144) and a flush path (140). The flush path, as shown in FIGS. 14c and 14d provides a conduit for flushing cleaning solution, saline, or other fluid when cleaning the deployment tool, and/or injecting saline or CO₂ mist to clear the field of view from blood. The rod (152) moves within a shell (150) that is bonded to the handle block (142). The length and orientation of rod and shell are determined by the procedure specifics. For less invasive access, the rod and shell are relatively long (>15 cm) to ensure the connector may reach the host vessel without the handle segments (146) interfering with the access points into the patient. The rod and shell may be curved to enable changing the angular pathway for inserting the connector into the host vessel. Alternatively, the rod and/or shell may be made malleable to enable the operator to tailor the deployment tool to his/her access viewpoint.

A compression spring (154) provides resistance to advancing the rod (152) relative to shell (150) and handle block (142) and ensures the resting position of the deployment tool is in the deflected state. The compression spring (154) is stiff enough such that with the trailing segment (18) of the fitting and the distal band (39) of the collar deflected, the deployment tool may be handed to the operator without having to manually hold the handle apart or worrying that the handle may accidentally become actuated and release the connector before it is appropriately positioned. Alternatively, a locking mechanism may be incorporated in the deployment tool to ensure the handle does not accidentally actuate.

The stabilizer (166) is bonded to the shell (150) and provides a support for the connector and defines the pivots for the toe deflector (164) and the heel deflector (162). The stabilizer also determines the angle at which the connector sits relative to the rod and shell of the deployment tool. For reverse insertion the stabilizer (166) is configured to orient the toe of the connector at an acute angle (<90 degrees) to the shell of the deployment tool. For perpendicular insertion, the stabilizer is configured to orient the toe of the connector at approximately 90 degrees to the shell. For acute insertion, the stabilizer is configured to orient the heel of the connector at an acute angle (<90 degrees) to the shell.

The toe deflector (164) and the heel deflector (162) are rotatably attached to the stabilizer (166) with pins (156). Intermediate linkages (158 and 160) connect the proximal ends of the heel deflector (162) and the toe deflector (164) to the rod (152) with a second compression spring (154) to orient the deflectors in the appropriate resting, “deflected” orientation when released. The intermediate linkages (158 and 160) and the associated compression spring (154) enable the offset deflection of the, toe deflector (164) from the heel deflector (162). As the heel deflector is actuated by squeezing the handles (146) the toe deflector (164) remains in the deflected, non-released position until the trailing segment (18) is fully released and the compression spring (154) is fully actuated such that movement of the rod engages the toe deflector linkage (160) which initiates the actuation of the toe deflector (164) and releases the distal band (39) of the collar. This two-staged release provides one additional benefit in that a tactile signal indicates the complete release of the trailing segment (18) and initiation of the release of the distal collar band (39). The toe deflector (164) provides another benefit in that it separates the ears (37) of the connector from engagement with the pins (170) once fully actuated to filly release the connector from the deployment tool and indicating completion of the angled anastomosis.

Once in place, the completed anastomosis is inspected for leakage. This may be done before and/or after an anastomosis at the other end of the graft (if required) is complete. At a minimum, an inspection of the angled anastomosis should be made when blood is flowing through graft (6). If leakage is detected, and it cannot be remedied by adjustment of the graft or collar, the anastomosis site may be packed until bleeding terminates, bioglue (e.g., as available through Cryolife in Kennesaw, Ga.) may be applied to the anastomosis, and/or a stitch of suture material may be applied.

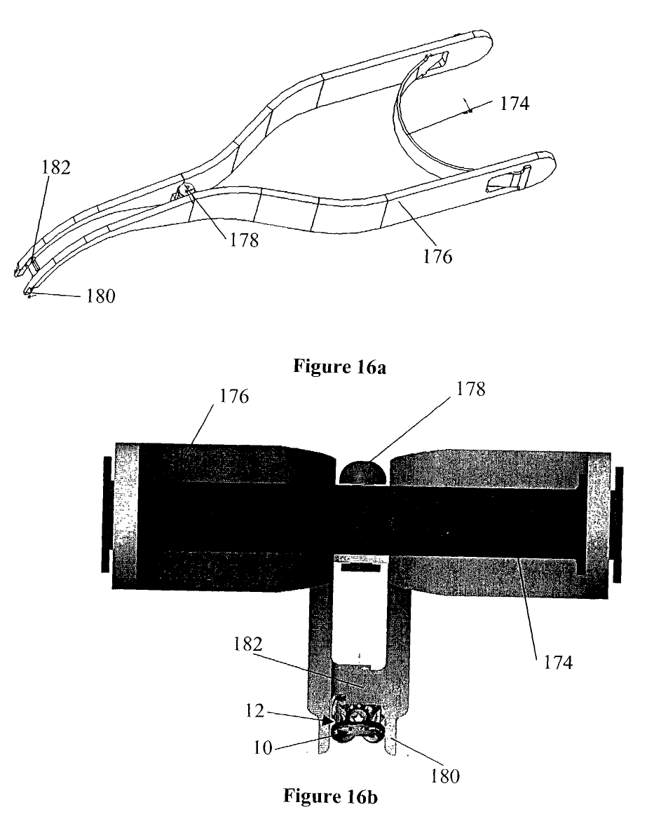

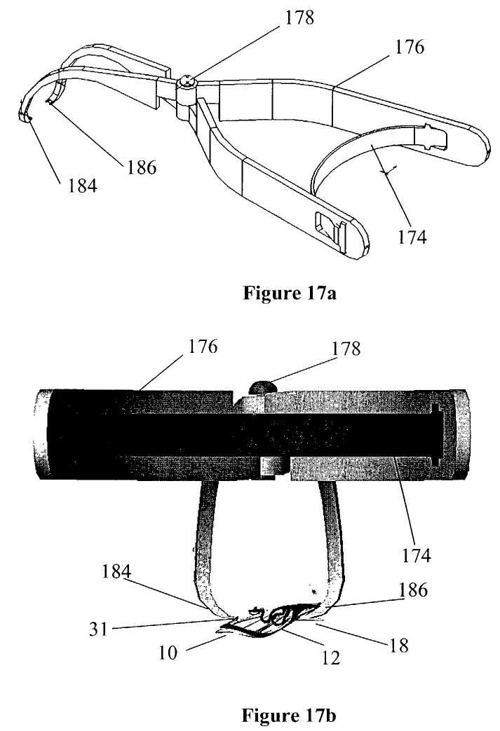

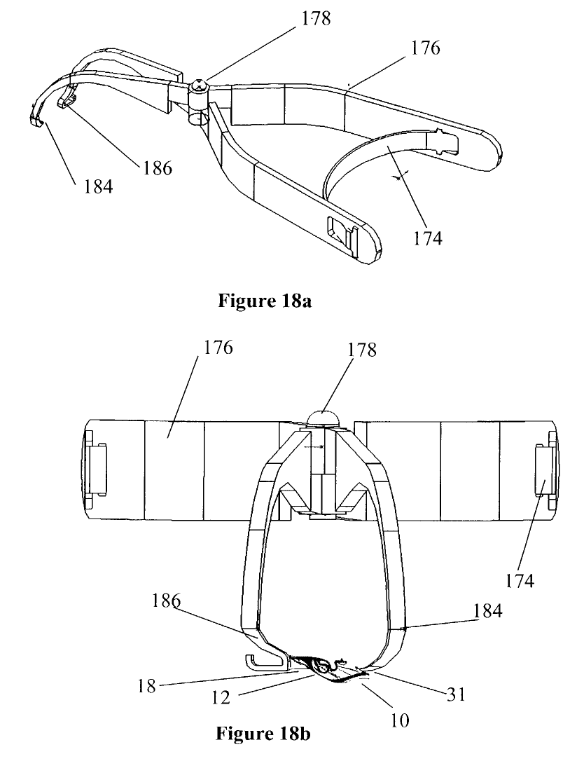

In extremely rare instances where these steps do not prove adequate, it may be necessary to reposition or remove the connector (4). FIGS. 16a and 16b show a repositioning tool designed to spread the sides of the collar distal band (39) and manipulate the connector such that tissue enters the gap between the lateral portions (20) of the fitting and the distal bad (39) of the collar. Once repositioned, the repositioning tool releases the collar. The repositioning tool has two handles (176) rotatably joined at a pivot pin (178) and with a spring (174). The functional end of the repositioning tool contains extensions (180) designed to fit within the edges of the distal band (39) and spread the distal band once actuated. A stabilization bar (182) is integrated with the extensions (180) and provides a surface to advance the connector once the distal band is spread open. FIGS. 17a and 17b show an extraction/repositioning tool whose active end contains a toe grasping rod (184) and a heel pusher (186) having similar engagement features as the toe deflector and heel deflector discussed above. The toe grasping rod deflects the distal band (39) of the collar while the heel pusher deflects the trailing segment of the fitting. This tool may be used to partially deflect the distal band and trailing segment to reposition the connector or fully deflect those components to remove the connector from the host vessel. FIGS. 18a and 18b show a removal tool that differs from the embodiment in FIGS. 17a and 17b in that the heel pusher (186) is curved to fully advance the trailing segment (18) of the fitting as the curved end is advanced into the wedge between the base (14) of the fitting and the trailing segment (18).

For less invasive approaches, bridging or endoscopic vein harvesting tools may be utilized to access the host vessel, expose the host vessel and stabilize the host vessel as the arteriotomy is created and the connector is deployed into the host vessel. Such devices include the SaphLITE® manufactured by Genzyme Surgical, Inc. for saphenous vein harvesting. This, and other such bridging devices, may be used to access peripheral host vessels through a small incision, and enable a less invasive approach to inserting angled connectors into the popliteal artery, femoral artery, iliac artery, etc. due to the features of the connector and accessory devices. The connector may also be used in conjunction with anastomosis isolation devices such as the eNclose® Anastomosis Assist Device manufactured by Novare Surgical, Inc. Such isolation devices clamp a region of the aorta and provide a membrane to prevent bleeding while the anastomosis is created. As such, the angled connector embodiments in this invention may readily be inserted through an incision created prior to or after deploying such isolation device and used to create the anastomosis.

Fabricating Connector Components

Now, returning to the elements of connector (4), optional inventive features and a manner of manufacture is described. A preferred manner of producing connector components according to the present invention is by machining tubing to include features that may be stressed and set into shape to produce connector elements like those depicted in FIGS. 1, 2, 3a, 3b, 4a, 4b, 6a, 6b, 6c, 6d, 7a, 7b, 8a, 8b, 9a, and 9b. Shapes so produced may be referred to as wireforms.

The machining may be accomplished by electron discharge machining (EDM), mechanically cutting, laser cutting or drilling, water-jet cutting or chemically etching. It is to be noted that portions of the connectors may be fabricated as a separate components and bonded by spot welding, laser welding or other suitable manufacturing process to form complete structures. Typically, after whatever cutting or forming procedure is employed, the material is set in a desired final shape. Where a metal is used, one or more flexure steps followed by heating will accomplish this. If the connector elements are made of alternate material such as a plastic or a composite, other forming procedures as would be apparent to one with skill in the art may be used.

Preferably, connector elements are made from a metal (e.g., titanium) or metal alloy (e.g., stainless steel or nickel titanium). Other materials such as thermoplastic (e.g., PTFE), thermoset plastic (e.g., polyethylene terephthalate, or polyester), silicone or combination of the aforementioned materials into a composite structure may alternatively be used. Also, connectors fabricated from nickel titanium may be clad with expanded PTFE, polyester, PET, or other material that may have a woven or porous surface. The fittings may be coated with materials such as paralyne or other hydrophilic substrates that are biologically inert and reduce the surface friction. To further reduce the surface tension, metallic or metallic alloy fittings may be bead blasted, chemically etched, and/or electropolished. Evidence suggests that electropolishing reduces platelet adhesion because of the smooth surface. Alternatively, the fittings may be coated with heparin, thromboresistance substances (e.g., glycoprotein IIb/IIIa inhibitors), antiproliferative substances (e.g., rapamycin), or other coatings designed to prevent thrombosis, hyperplasia, or platelet congregation around the attachment point between the bypass graft and the host vessel. Alternatively, a material such as platinum, gold, tantalum, tin, tin-indium, zirconium, zirconium alloy, zirconium oxide, zirconium nitrate, phosphatidyl-choline, or other material, may be deposited onto the fitting surface using electroplating, sputtering vacuum evaporation, ion assisted beam deposition, vapor deposition, silver doping, boronation techniques, a salt bath, or other coating process.

A still further improvement of the fittings is to include beta or gamma radiation sources on the end-side fittings. A beta or gamma source isotope having an average half-life of approximately 15 days such as Phosphorous 32 or Palladium 103 may be placed on the base and/or petals of the end-side fitting using an ion-implantation process, chemical adhesion process, or other suitable method. Further details as to optional treatments of connectors according to the present invention are described in 10.00. Of course, connector fitting (10) and any associated collar (12) may be made differently. To avoid electrolytic corrosion, however, dissimilar metals should not be used.

Preferably, NiTi (Nitinol) tubing or flat stock is used to produce connector components. Irrespective of material format, a preferred alloy includes a 54.5-57% Ni content, and a remainder Ti by weight (less minor amounts of C, O, Al, Co, Cu, Fe, Mn, No, Nb, Si and W) is used. Such alloy has an A_{f} for at about −10 to −15° C. Consequently, for typical handling and in use, the material will exhibit superelastic properties as is most desired.

Still, it is contemplated that connectors according to the present invention may utilize thermoelastic or shape memory characteristics instead, wherein the material of either or both fitting (10) and connector (12) change from a martensitic state to an austenitic state upon introduction to an anastomosis site and exposure to a sufficiently warm environment. Taking advantage of the martensitic state of such an alloy will ease deflecting rear segment (18) and distal band (39) and maintaining their positions until placement.

Utilizing either thermoelastic or superelastic properties makes for a connector that can have certain members stressed to a high degree and return without permanent deformation from a desired position. However, it is contemplated that either or both fitting (10) and collar (12) may be made of more typical materials such as stainless steel or plastic. For fitting (10), this is feasible in view of the manner in which rear segment (18) is displaced for insertion into a host vessel. Hinge section (28) permits designs in which the stress applied by torsion is lower that applied in simply deflecting a rear petal or segment as shown and described in U.S. and foreign patents and applications entitled, “Improved Anastomosis Systems”, U.S. patent application Ser. No. 09/730,366; “End-Side Anastomosis Systems”, PCT Publication No. WO 01/41653; “Advanced Anastomosis Systems (II)” U.S. patent application Ser. No. 09/770,560.

This being said, the tube stock used to prepare distal connector fitting preferably has an outer diameter between 0.080 and 0.240 in (2 to 6 mm) and a wall thickness between 0.004 and 0.010 in (0.1 to 0.25 mm). Slightly larger diameter stock (or end product) will be used for each matching collar. The stock thickness for NiTi material used to form collars will typically have a wall thickness between about 0.004 in and about 0.010 in. Especially, for fitting (10) where it is possible to use thin stock in view of strength requirements, this will be preferred in order to minimally obstruct blood flow past the fitting. Larger connector components will typically be made of thick stock to account for increased stiffness required of such configurations relative to smaller ones.

The invention has been described and specific examples or variations of the invention have been portrayed. The use of those specific examples is not intended to limit the invention in any way. In all, it is to be understood that each of the features described in connection with the various connector components and projections for forming the same may be mixed and matched to form any number of desirable combinations. Further, it is contemplated that additional details as to the use or other aspects of the system described herein may be drawn from Abstract, Field of the Invention, Background of the Invention, Summary of the Invention, Brief Description of the Drawings, the Drawings themselves and Detailed Description and other background that is intended to form part of the present invention, including any of the patent applications cited above, each of which being incorporated by reference herein in its entirety for any purpose. Also, to the extent that there are variations of the invention which are within the spirit of the disclosure and are equivalent to features found in the claims, it is the intent that the claims cover those variations as well. All equivalents are considered to be within the scope of the claimed invention, even those which may not have been set forth herein merely for the sake of relative brevity. Finally, it is contemplated that any single feature or any combination of optional features of the inventive variations described herein may be specifically excluded from the invention claimed and be so-described as a negative limitation.

What is claimed is:

1. An anastomosis connector system, comprising:
a fitting comprising a base adapted for attachment to a graft, a leading segment adapted for introduction into a host vessel, and a trailing segment having a proximal end and a distal end, wherein the proximal end of the trailing segment is integrally attached along a torsional region which extends between the base and the leading segment,
wherein the distal end of the trailing segment is adapted to extend through a first area of the graft such that the graft is at least partially secured to the fitting, the trailing segment being deflectable about the torsional region from a first position to a second position such that at least the leading segment and the trailing segment can be advanced into the host vessel.

2. The system of claim 1 wherein the trailing segment is adapted to return to the first position after being advanced into the host vessel such that retraction from the host vessel is inhibited.

3. The system of claim 1 wherein the distal end of the trailing segment extends through a puncture defined in the graft.

4. The system of claim 1 wherein the fitting further comprises at least one fastener for securing a second area of the graft to the fitting.

5. The system of claim 4 wherein the fastener comprises at least one pin adapted to hold the graft to the fitting during introduction into the host vessel.

6. The system of claim 1 further comprising a collar which is adapted to secure the graft to the host vessel between the fitting and the collar.

7. The system of claim 6 wherein the collar comprises an elongate heel segment which defines an opening adapted to receive the trailing segment for securing the collar to the fitting.

8. The system of claim 6 wherein the collar further comprises a collar tab adapted to interface with a complementary tab located on the fitting for securing the graft between the fitting and the collar.

9. The system of claim 8 wherein the collar further comprises at least one side spring member which extends from a heel portion of the collar to the collar tab.

10. The system of claim 8 wherein the side spring member is formed into a looped configuration.

11. The system of claim 6 wherein the collar further comprises at least one expansion spring member which is biased to compress the collar about the fitting.

12. The system of claim 11 wherein the expansion spring member has an undulating pattern comprising a middle undulation and at least two adjacent side undulations, wherein the middle undulation has a length which is shorter than a length of the side undulations.

13. The system of claim 6 wherein the collar further comprises a distal band member which extends around the graft from a heel portion and is adapted to urge the graft against the collar.

14. The system of claim 1 wherein the fitting comprises a biocompatable material selected from the group consisting of stainless steel, titanium, and titanium alloy.

15. The system of claim 14, wherein the titanium alloy comprises NiTi.

16. The system of claim 6 wherein the collar comprises a biocompatable material selected from the group consisting of stainless steel, titanium and titanium alloys.

17. The system of claim 16 wherein the titanium alloy comprises NiTi.

18. The system of claim 1 wherein a superelastic effect returns the trailing segment from the second position to the first position.

19. The system of claim 1 wherein a thermoelastic or shape-memory effect returns the trailing segment from the second position to the first position.

20. The system of claim 1 further comprising an instrument adapted to hold the fitting for deployment by deflecting the trailing segment.

21. The system of claim 1 wherein the fitting further comprises a coating selected from the group consisting of biologically inert and biologically reactive materials.

22. The system of claim 1 wherein the fitting comprises a wireform.

23. The system of claim 22 wherein the wireform is produced by a method selected from the group consisting of electron discharge machining, mechanical cutting, laser cutting, laser drilling, water-jet cutting, and chemically etching.

24. The system of claim 22 wherein the wireform is comprised of a singular integral structure.

25. The system of claim 22 wherein the wireform is produced from tubing stock or flat stock from which material is removed.

26. The system of claim 25 wherein the tubing stock or flat stock has a wall thickness between about 0.004 in and about 0.010 in.

27. The system of claim 6 wherein the collar further comprises a coating selected from the group consisting of biologically inert and biologically reactive materials.

28. The system of claim 6 wherein the collar comprises a wireform.

29. The system of claim 28 wherein the wireform is produced by a method selected from the group consisting of electron discharge machining, mechanical cutting, laser cutting, laser drilling, water-jet cutting, and chemically etching.

30. The system of claim 28 wherein the wireform is comprised of a singular integral structure.

31. The system of claim 28 wherein the wireform is produced from tubing stock or flat stock from which material is removed.

32. An integral anastomosis connector system, comprising:
a fitting comprising a base adapted for attachment to a graft, a leading segment adapted for introduction into a host vessel, and a trailing segment having a proximal end and a distal end;
a collar comprising a distal band member which is extendable around the graft from an elongate heel segment, the distal band member being adapted to urge the graft against the fitting;
a first hinge defined between the leading segment and the base, wherein the first hinge is adapted to deflect the leading segment relative to the base when securing the graft to the fitting; and
a second hinge defined between the collar and the base.

33. The system of claim 32 further comprising at least one pin extending from the fitting, the at least one pin being adapted to secure an everted end of the graft to the fitting.

34. The system of claim 32 wherein each of the first and second hinges has a variable stiffness and spring constant.

35. The system of claim 32 wherein the heel segment is adapted to compress the host vessel against the trailing segment.

36. The system of claim 32 wherein the distal end of the trailing segment is adapted to extend through a first area the graft such that the graft is at least partially secured to the fitting.

37. The system of claim 32 further comprising at least one lateral portion extending between the proximal end of the trailing segment to the leading segment.

38. The system of claim 32 wherein the trailing segment is deflectable from a first position to a second position such that at least the leading segment and the trailing segment can be advanced into the host vessel.

39. The system of claim 38 wherein the trailing segment is adapted to return to the first position after being advanced into the host vessel such that retraction from the host vessel is inhibited.

40. A connector loading tool system for preparing an anastomosis connector assembly, the system comprising:
a loading tool base;
an outer frame cartridge comprising a flex region having an integral hinge about which a plurality of pins are attached, the pins being adapted to engage an expandable anastomotic connector collar, and an interlock adapted to secure the outer frame cartridge to the loading tool base; and
an inner frame cartridge having a handle with a snap defined thereon for engaging a slidable mount positioned on the loading tool base in apposition to the outer frame cartridge, the inner frame cartridge being adapted to engage an anastomotic fitting which is configured to receivingly engage the expandable connector collar;
wherein the loading tool base is configured to advance the inner frame cartridge towards the outer frame cartridge until the expandable connector collar is engaged with the fitting.

41. The tool system of claim 40 wherein the loading tool base further comprises a lever adapted to expand the connector collar when actuated.

42. The tool system of claim 40 wherein the outer frame cartridge further comprises a removable mating insert adapted to stabilize and mate with the pins.

43. The tool system of claim 40 further comprising an elongate pusher tool adapted to secure the connector collar to the fitting.

44. The tool system of claim 40 wherein the connector collar expands via an expansion spring to receivingly engage the fitting therewithin.

45. The tool system of claim 44 wherein the connector collar expands between 0.070 in to 0.150 in via the expansion spring.

46. The tool system of claim 44 wherein the connector collar engages the fitting via complementary tabs positioned on the fitting.

47. An anastomosis deployment tool assembly, comprising:
a handle block having at least one actuator positioned therewithin;
an elongate shell attached at a distal end of the handle block, the shell defining a lumen therethrough;
an elongate rod having a proximal end, a distal end, and a length therebetween, the proximal end being positioned within the handle block and pivotally connected to the at least one actuator such that movement of the actuator correspondingly advances the rod within the shell lumen;
a biasing mechanism located within the handle block which is adapted to bias the rod in a non-advanced position relative to the shell;
a stabilizer attached to a distal end of the shell, wherein the stabilizer defines a first and a second pivot; and
a first deflector rotatably attached to the first pivot of the stabilizer and a second deflector rotatably attached to the second pivot of the stabilizer;
wherein advancing the rod distally through the shell actuates movement of the first deflector and the second deflector.

48. The deployment tool assembly of claim 47 wherein the actuator comprises at least one linkage having a proximal end attached to at least one handle and a distal end attached to the rod, wherein the linkage passes through an opening defined by the handle block.

49. The deployment tool assembly of claim 48 wherein the handle is pivotally attached to the handle block.

50. The deployment tool assembly of claim 47 wherein the rod defines a lumen therethrough such that the distal end of the rod is in fluid communication with the proximal end of the rod.

51. The deployment tool assembly of claim 47 wherein the rod and shell are adapted to conform to an arbitrary shape defined along each length.

52. The deployment tool assembly of claim 47 wherein the biasing mechanism comprises a compression spring positioned within the handle block coaxially about the rod.

53. The deployment tool assembly of claim 47 further comprising a locking mechanism for preventing movement of the rod relative to the shell.

54. The deployment tool assembly of claim 47 wherein each of the first and the second deflectors are attached to the stabilizer via pins.

55. The deployment tool assembly of claim 47 wherein the first deflector is actuated via a first intermediate linkage and the second deflector is actuated via a second intermediate linkage, each of the intermediate linkages being connected to the distal end of the rod.

56. The deployment tool assembly of claim 47 wherein the first deflector is actuated prior to the second deflector being actuated.

57. The deployment tool assembly of claim 47 wherein the first deflector and the second deflector are actuated simultaneously.

58. An anastomosis connector placement tool comprising:
a handle portion; and
an active portion having at least a first and a second extension extending from the handle portion,
wherein the first extension is adapted to engage a trailing segment of an anastomosis connector and the second extension is adapted to engage an expandable collar of the connector and manipulate the collar via actuation of the handle portion.

59. The placement tool of claim 58 further comprising a stabilization bar integrated between the extensions.

60. The placement tool of claim 58 wherein the handle portion is rotatable relative to the active portion via a pivot connecting the portions.

61. The placement tool of claim 58 further comprising a spring connected between two members of the handle portion for providing a biasing force to the active portion.
